(19) United States
(12) Patent Application Publication
(10) Pub. No.: US 2005/0054027 A1
(43) Pub. Date: Mar. 10, 2005

(54) Modulators of transmembrane protease serine 6

(75) Inventors: Jennifer Harris, San Diego, CA (US); Aaron Shipway, Cardiff, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 10/933,666

(22) Filed: Sep. 3, 2004

Related U.S. Application Data
(60) Provisional application No. 60/501,301, filed on Sep. 9, 2003.

(51) Int. Cl. C12Q 1/37; C12Q 1/18

(52) U.S. Cl. 435/23; 435/32

(57) ABSTRACT

This invention provides novel methods for identifying modulators of transmembrane protease serine 6 (TMPRSS6). The methods comprise screening test agents for ability to modulate proteolysis of a pathogenic toxin substrate or a synthetic peptide substrate of TMPRSS6. The methods can further comprise screening the identified modulating agents for ability to inhibit infections of pathogens. Also provided in the invention are methods and pharmaceutical compositions for treating infections of pathogens whose toxins are proteolytically activated by TMPRSS6.

### CROSS-REFERENCE TO RELATED APPLICATIONSU

This application claims the benefit of priority under 35 U.S.C. §119(e) to U.S. Provisional Patent Application No. 60/501,301, filed Sep. 9, 2004. The disclosure of the priority application is incorporated herein by reference in its entirety and for all purposes.

### FIELD OF THE INVENTION

The present invention generally relates to a host transmembrane serine protease that could play an important role in proteolytic activation of toxins from a number of pathogens. Provided herein are methods for identifying modulators (e.g., inhibitors) of this protease and therapeutic applications of such modulators.

### BACKGROUND OF THE INVENTION

Many pathogenic toxins are synthesized in a proform and require proteolytic activation before they can intoxicate cells they infect. Often, they are cleared and activated by eukaryotic proteases. For example, anthrax toxin protective antigen is activated by a cell surface protease with the sequence specificity and catalytic properties of furin, a calcium-dependent serine protease that recognizes the sequence Arg-X—X-Arg. See, e.g., Beauregard et al., Cell. Microbiol., 2: 251-58, 2000; Klimpel et al., Proc Natl Acad Sci USA. 89: 10277-81, 1992; and Molloy et al., J Biol Chem. 267: 16396-402, 1992. Furin also mediates activation of proteins of many other pathogens, e.g., pseudomonas exotoxin of Pseudomonas aeruginosa (Chiron et al., J Biol Chem. 272(50):31707-11, 1997; and Inocencio et al., J Biol Chem. 269: 31831-5, 1994); diphtheria toxin of Corynebacterium diphtheriae (Chiron et al., Mol Microbiol. 22(4):769-78, 1996; Chiron et al., J Biol Chem. 269(27):18167-76, 1994; and Tsuneoka et al., J Biol Chem. 268(35):26461-5, 1993); and shiga toxin of Shigella dysenteriae (Garred et al., J Biol Chem. 270(18):10817-21, 1995).

There is a need in the art for finding and characterizing more proteases that are involved in activation of pathogenic proteins, and for developing specific inhibitors of such proteases in treating infection of these pathogens. The instant invention fulfills this and other needs.

### SUMMARY OF THE INVENTION

The present invention relates to methods of screening for modulators (e.g., inhibitors) of transmembrane protease serine 6 (TMPRSS6), and methods for using the modulators to treat infections of pathogens that require proteolytic activation of their proteins by furin-like host proteases. In one aspect, the invention provides methods for identifying agents that inhibit proteolytic activation of a toxin of a pathogen. The methods entail (a) examining proteolysis of the toxin by transmembrane protease serine 6 (TMPRSS6), or an enzymatic fragment of TMPRSS6, in the presence of test agents; and (b) identifying an agent that inhibits proteolysis of the toxin by TMPRSS6.

Some of the methods are directed to a pathogen that is selected from the group consisting of Bacillus anthracis, Pseudomonas aeruginosa, Corynebacterium diphtheriae, and Shigella dysenteriae. In some of the methods, the toxin employed is anthrax protective antigen of Bacillus anthracis. In some methods, the toxin employed is pseudomonas exotoxin of Pseudomonas aeruginosa. In some other methods, the toxin used is diphtheria toxin of Corynebacterium diphtheriae. Some other methods employ shiga toxin of Shigella dysenteriae.

In some of the methods, the enzymatic fragment of TMPRSS6 used contains its catalytic domain. In some methods, the TMPRSS6 protease employed is encoded by a polynucleotide having accession number AY190317, AB048797 or AB048796. In some of the methods, proteolysis of the toxin can be examined by polyacrylamide gel electrophoresis.

In another aspect, the invention provides methods for identifying agents that inhibit infection of a pathogen. The methods entails (a) assaying protease activity of transmembrane protease serine 6 (TMPRSS6), or an enzymatic fragment of TMPRSS6, in the presence of test agents to identifying one or more modulating agents that inhibit the protease activity of TMPRSS6; and (b) examining the modulating agents for ability to inhibit or clear infection of the pathogen.

Some of these methods are directed to a pathogen selected from the group consisting of Bacillus anthracis, Pseudomonas aeruginosa, Corynebacterium diphtheriae, and Shigella dysenteriae. In some of the methods, an enzymatic fragment of TMPRSS6 that contains the catalytic domain is employed. In some methods, the TMPRSS6 protease employed is encoded by a polynucleotide having accession number AY190317, AB048797 or AB048796.

In some of the methods, the modulating agents are examined with an animal infected with the pathogen. In some of the methods, the protease activity of TMPRSS6 is assayed with a toxin of the pathogen. In some other methods, the protease activity of TMPRSS6 is assayed with a synthetic peptide substrate. In these methods, sequence at P4-P1 positions of the peptide substrate can be selected from the group consisting of RKFK, RAFK, AKFK, and AAFK. In some methods, the peptide substrate is labeled with a fluorogenic compound. In some of these methods, the fluorogenic compound employed is 7-amino-4-carbamoylmethylcoumarin or 7-amino-3-carbamoylmethyl-4-methylcoumarin.

In another aspect of the invention, methods for treating infection of a pathogen in a subject are provided. The methods entail administering to the subject a pharmaceutical composition comprising an effective amount of an agent that inhibits the protease activity of transmembrane protease serine 6 (TMPRSS6). In some of these methods, the TMPRSS6 protease employed is encoded by a polynucleotide having accession number AY190317, AB048797 or AB048796. Some of the methods are directed to treating infection of a pathogen selected from the group consisting of Bacillus anthracis, Pseudomonas aeruginosa, Corynebacterium diphtheriae, and Shigella dysenteriae.

In some of the methods, the TMPRSS6-inhibiting agent employed is camostat mesylate. In some other methods, the TMPRSS6-inhibiting agent is identified in accordance with claim 1. In some methods, the agent is identified in accordance with claim 10. In some methods, the TMPRSS6-inhibiting agent employed inhibits proteolysis of anthrax protective antigen of Bacillus anthracis. In some other methods, the agent employed inhibits proteolysis of Corynebacterium diphtheriae.

A further understanding of the nature and advantages of the present invention may be realized by reference to the remaining portions of the specification and claims.

### BRIEF DESCRIPTION OF THE DRAWINGS

FIG. 1 shows determination of optimal substrate specificity of TMPRSS6 at the P1-P4 positions.

FIG. 2 shows determination of optimal substrate specificity of TMPRSS6 at the P1′-P4′ positions.

FIG. 3 shows kinetics of digestion of different substrates by TMPRSS6.

FIG. 4 shows inhibition of TMPRSS6 protease activity by camostat mesylate.

FIG. 5 shows similarity between substrate specificity of TMPRSS6 and furin recognition sites in several known bacterial toxins.

FIG. 6 shows cleavage of anthrax protective antigen by TMPRSS6.

FIG. 7 shows cleavage of diphtheria toxin by TMPRSS6.

FIG. 8 shows enhanced cytotoxicity of diphtheria toxin cleaved by TMPRSS6.

### DETAILED DESCRIPTION

The present invention is predicated in part on the discovery by the present inventors that transmembrane protease serine 6 (TMPRSS6) has similar substrate specificity to that of furin, that several bacterial toxins were proteolytically digested by TMPRSS6, and that toxins thus digested displayed enhanced cytotoxicity (see Examples below). In accordance with these discoveries, the invention provides methods for identifying modulators (e.g., inhibitors) of TMPRSS6. Inhibitory modulators thus identified can be used to inhibit proteolytic activation of pathogenic proteins (e.g., bacterial toxins), and to treat infections of the pathogens in a subject.

To identify novel modulators of TMPRSS6, test agents can be first screened for ability to bind to TMPRSS6, its fragments, variants or analogs. Agents thus identified can be then further examined for activity in modulating (e.g., inhibiting) the enzymatic activity of TMPRSS6. Alternatively, test agents can be directly subject to screening for ability to modulate proteolysis of a substrate (e.g., a bacterial toxin or a synthetic peptide substrate) by TMPRSS6. Typically, the test agents are screened for ability to inhibit TMPRSS6 protease activity. However, modulators that enhance the protease activity can also be screened for with methods of the present invention. Further, once an agent has been identified to modulate (e.g., inhibit) TMPRSS6 protease activity, it can be further tested for ability to inhibit infection of the pathogen in a subject, e.g., with an animal model.

The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more molecules (e.g., a test agent and a polypeptide) or combining molecules and cells (e.g., a test agent and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology.

A “host cell,” as used herein, refers to a prokaryotic or eukaryotic cell that contains heterologous DNA that has been introduced into the cell by any means, e.g., transfection, electroporation, calcium phosphate precipitation, microinjection, transformation, viral infection, and/or the like.

The term “sequence identity” in the context of two nucleic acid sequences or amino acid sequences refers to the residues in the two sequences which are the same when aligned for maximum correspondence over a specified comparison window. A “comparison window” refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, e.g., TMPRSS6, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., as measured by BLASTN (or CLUSTAL, or any other available alignment software) using default parameters.

A “substantially identical” nucleic acid or amino acid sequence refers to a nucleic acid or amino acid sequence which comprises a sequence that has at least 90% sequence identity to a reference sequence using the programs described above (preferably BLAST) using standard parameters. The sequence identity is preferably at least 95%, more preferably at least 98%, and most preferably at least 99%. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “modulate” with respect to a reference protein or its fragment refers to a change in one or more biological activity of the protein. For example, modulation may cause an increase or a decrease in expression level of the reference protein, enzymatic modification of the protein, binding characteristics (e.g., binding to a substrate), enzymatic activities of the protein, or any other biological, functional, or immunological properties of the reference protein.

Modulation of a reference protein can be up-regulation (i.e., activation or stimulation) or down-regulation (i.e. inhibition or suppression). The mode of action of a modulator on a reference protein can be direct, e.g., through binding to the protein or to genes encoding the protein, or indirect, e.g., through binding to and/or modifying (e.g., enzymatically) another molecule which otherwise modulates the reference protein.

The term “operably linked” refers to a functional relationship between two or more polynucleotide (e.g., DNA) segments. Typically, it refers to the functional relationship of a transcriptional regulatory sequence to a transcribed sequence. For example, a transcription regulatory sequence is operably linked to a coding sequence if it modulates the transcription of the coding sequence in an appropriate host cell or other expression system. Generally, transcriptional regulatory sequences that are operably linked to a transcribed sequence are physically contiguous to the transcribed sequence, i.e., they are cis-acting. However, some transcription regulatory sequences, such as enhancers or response elements, need not be physically contiguous or located in close proximity to the coding sequences whose transcription they enhance.

“Protease” typically refers to an enzyme that degrades proteins or peptides by hydrolyzing peptide bonds between amino acid residues. In some embodiments, proteases, also known as proteinases, peptidases, or proteolytic enzymes, are used to cleave non-peptide substrates. There are various types of proteases including serine proteases, threonine proteases, metalloproteases, cysteine proteases, aspartyl proteases, and the like. Many proteases are non-specific in their activity, meaning that they digest proteins to peptides and/or amino acids. Other proteases are more specific, cleaving only a particular protein or only between certain predetermined amino acids. Still other proteases have optimal sequences that they cleave preferentially over others.

A protease recognition site typically comprises one or more non-prime positions and one or more prime positions, each of which positions is occupied by a substrate moiety. The prime and non-prime positions flank the protease cleavage site, with the non-prime positions being defined as being on the amino-terminal side of the cleavage site, and the prime positions being on the carboxy-terminal side of the cleavage site.

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression vector” or simply an “expression vector” is a nucleic acid construct, generated recombinantly or synthetically, that has control elements that are capable of affecting expression of a structural gene that is operably linked to the control elements in hosts compatible with such sequences. Expression vectors include at least promoters and optionally, transcription termination signals. Typically, the recombinant expression vector includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression vector.

The term “subject” includes human and non-human animals, e.g., non-human mammals.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

II. TMPRSS6 Polynucleotides and Polypeptides

Biochemical properties of TMPRSS6 were characterized by the present inventors as described in the Examples below. Polynucleotide sequence of TMPRSS6 is known in the art (accession number AY190317). Substantially identical sequences were also cloned and reported by Kim et al., Biochimica et Biophysica Acta 1518: 204-209, 2001 (accession numbers AB048796 and AB048797). Corresponding amino acid sequence of AY190317 is nearly 100% identical to that encoded by AB048796 except for a few amino acid differences in the pro-region (N-terminal to the catalytic domain) and one amino acid in the catalytic region.

Any of these known sequences, or corresponding sequences from commercially available human cDNA libraries, can be employed to produce the enzyme. For example, as described in the Examples, TMPRSS6 or its catalytic fragment can be cloned into an expression vector such as pFastBacl (Invitrogen), and overexpressed in eukaryotic or prokaryotic host cell, e.g., the Sf9 cells (Invitrogen). Purification of the overexpressed protein can be carried out using routinely practiced methods for protein purification, e.g., as described in the Examples below.

Other expression vectors and host systems can also be used for obtaining TMPRSS6 polypeptides or variants that are suitable for practicing the present invention. For example, a variety of mammalian expression vectors may be used to express recombinant TMPRSS6 in mammalian cells. Commercially available mammalian expression vectors include, e.g., pCI Neo (Promega, Madison, Wis., Madison Wis.), pMAMneo (Clontech, Palo Alto, Cailf.), pcDNA3 (InVitrogen, San Diego, Calif.), pMClneo (Stratagene, La Jolla, Calif.), pXT1 (Stratagene, La Jolla, Calif.), pSG5 (Stratagene, La Jolla, Calif.), EBO-pSV2-neo (ATCC 37593), pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565). Yeast expression vectors may also be used to express TMPRSS6. Examples of commercially available yeast expression vectors and hosts include pPICz(alpha) in Pichia pastoris or pYES2-DEST52 (Invitrogen) in Sacromyces cerivisea. Other than mammalian and yeast expression vectors, many bacterial expression vectors can also be used to express recombinant TMPRSS6 or its fragments. Examples of commercially available bacterial expression vectors suitable for practicing the present invention include pET vectors (Novagen, Inc., Madison Wis.), pQE vectors (Qiagen, Valencia, Calif.) and pGEX (Pharmacia Biotech Inc., Piscataway, N.J.).

Host cells to harbor the expression vectors may be prokaryotic or eukaryotic, including, e.g., bacteria cells such as E. coli, fungal cells such as yeast, mammalian cell lines of human, bovine, porcine, monkey or rodent origin, and insect cells such as Drosophila S2 (ATCC CRL-1963) and silkworm Sf9 (ATCC CRL-1711). Additional cell lines derived from mammalian species that may be suitable for the present invention are also commercially available. These include, e.g., CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, and HEK-293 (ATCC CRL 1573).

The expression vector may be introduced into host cells via any one of a number of techniques including, e.g., transformation, transfection, protoplast fusion, lipofection, and electroporation. The expression vector-containing cells are clonally propagated and individually analyzed to determine whether they produce TMPRSS6. Identification of TMPRSS6-expressing host cell clones may be done by several means, including but not limited to polyacrylamide gel electrophoresis, immunological reactivity with anti-TMPRSS6 antibodies, and the presence of host cell-associated TMPRSS6-like proteolytic activity.

Expression of TMPRSS6 may also be performed using in vitro produced synthetic mRNA. Synthetic mRNA or mRNA isolated from TMPRSS6-producing cells can be efficiently translated in various cell-free systems, e.g., wheat germ extracts and reticulocyte extracts. Expression can also be performed in cell based systems, e.g., microinjection into frog oocytes.

To screen for modulators of TMPRSS6, either an intact TMPRSS6, or its fragments, analogs, polypeptides with substantial identical sequences, or functional derivatives can be used. If a TMPRSS6 fragment is used, the fragment typically retains its substrate-binding and/or serine protease activity. For example, as described in the Examples below, TMPRSS6 to be employed in the screening can be a recombinant fragment containing its catalytic domain. Fusion proteins containing a TMPRSS6 fragment or analog can also be used for the screening of test agents. Functional derivatives of TMPRSS6 usually have amino acid deletions and/or insertions and/or substitutions while maintaining one or more of the biological activities (e.g., substrate binding or proteolytic activity), and therefore can also be used in practicing the screening methods of the present invention.

A functional derivative of TMPRSS6 can be prepared from a naturally occurring or recombinantly expressed TMPRSS6 polypeptides by proteolytic cleavage followed by conventional purification procedures known to those skilled in the art. Alternatively, the functional derivative can be produced by recombinant DNA technology by expressing only fragments of TMPRSS6 that retain one or more of its biological activities, e.g., its catalytic activity as exemplified in the Examples below.

III. Pathogenic Proteins or Synthetic Substrates

Many substrates can be employed in the screening for modulators of TMPRSS6 protease activity. These include both natural occurring proteins from pathogens (e.g., bacteria) and synthetic peptide or polypeptide substrates. As detailed in the Examples, the present inventors discovered that TMPRSS6 exhibits similar substrate specificity to that of furin, a protease known to play a role in proteolytic activation of several bacterial toxins, viral proproteins, as well as proproteins of some serum proteins, growth factors, cell surface receptors and extracellular matrix proteins (see Rockwell et al., Chem Rev. 102: 4525-48, 2002).

For example, it was shown that furin and additional cellular proteases proteolytically activate a few bacterial toxins in eukaryotic cells. Furin processes proproteins of bacterial toxins such as anthrax protective antigen of Bacillus anthracis (Molloy et al., J Biol Chem. 267: 16396-402, 1992); pseudomonas exotoxin of Pseudomonas aeruginosa (Inocencio et al., J Biol Chem. 269: 31831-5, 1994); diphtheria toxin of Corynebacterium diphtheriae (Chiron et al., J Biol Chem. 269: 18167-76, 1994); shiga toxin of Shigella dysenteriae (Garred et al., J Biol Chem. 270: 10817-21, 1995); aerolysin of Aeromonas hydrophila (Abrami et al., J Biol Chem. 273: 32656-61, 1998); and alpha toxin of Clostridium septicum (Gordon et al., Infect Immun. 4130-4, 1997). Other than furin, there are other cellular proteases that are involved in activation of these bacterial toxins. For example, it was also known that furin-deficient cells were resistant to Pseudomonas exotoxin, but were sensitive to Diphtheria toxin and anthrax protective antigen (Gordon et al., Infect Immun. 63: 82-7, 1995).

Other than bacterial toxins, furin and furin-like proteases are also involved in proteolytic activation of some viral proteins. These include, e.g., the glycoprotein gp63 of HTLV-II (Hasegawa et al., AIDS Res Hum Retroviruses 18:1253-60, 2002); the E2 receptor-binding protein of Semliki Forest virus (Zhang et al., J Virol. 77(5):2981-9, 2003); and IL-18 binding protein of Molluscum contagiosum virus (Xiang et al., J Virol. 77(4):2623-30, 2003).

Due to the similarity between substrate specificity of furin and TMPRSS6 as characterized herein, these natural proteins from pathogens could be natural substrates of TMPRSS6. Just like furin, TMPRSS6 could also take part in regulating activities of these pathogenic proteins. Indeed, among the bacterial toxin substrates of furin, anthrax protective antigen and diphtheria toxin have been exemplified by the present inventors to be also digested by TMPRSS6. Moreover, the toxins thus cleaved by TMPRSS6 showed enhanced cytotoxicity to cells (see the Examples below).

Thus, these bacterial toxins or viral proteins could all be suitable in the present invention to monitor proteolytic activity of TMPRSS6. These proteins can be obtained from commercial suppliers or recombinantly produced. For example, anthrax protective antigen and diphtheria toxin are available from, e.g., List Biological Laboratories, Inc., Campbell, Calif. Also, these pathogenic proteins have all been characterized in the art, and their sequences disclosed. Therefore, other than commercial sources, these pathogenic proteins can also be produced by recombinant means.

In addition to the natural pathogenic proteins, synthetic peptides or polypeptides can also be used as substrates of TMPRSS6 in the screening. As exemplified in the Examples below, a synthetic peptide or polypeptide substrate usually has one or more substrate moieties. A substrate moiety can be any amino acid, peptide, protein, non-peptide moiety, small molecule, organic molecules, inorganic moiety, or the like. In some preferred embodiments, the substrates is labeled fluorescently, e.g., with a coumarin compound. Examples of coumarin compounds include 7-amino-4-carbamoylmethylcoumarin (ACC), 7-amino-4-methylcoumarin (AMC), and 7-amino-3-carbamoylmethyl-4-methylcoumarin, and the like. The coumarin compounds are available either commercially (e.g., from Sigma and Molecular Probes catalogs) or using various synthetic protocols known to those of skill in the art. Synthesis of substrates labeled with a coumarin compound has been described in detail in the art, e.g., WO 03/029823.

IV. Screening for TMPRSS6-Inhibiting Agents

Utilizing TMPRSS6 polynucleotides and polypeptides as well as the substrates described above, the present invention provides methods of screening for novel modulators of TMPRSS6 and methods of inhibiting proteolytic activation of pathogenic proteins. Various biochemical and molecular biological techniques or assays well known in the art can be employed to practice the present invention. Such techniques are described in, e.g., Sambrook et al., Molecular Cloning: A Laboratory Manual, Cold Spring Harbor Press, N.Y., 3^{rd} Ed (2000); and Ausubel et al., Current Protocols in Molecular Biology, John Wiley & Sons, Inc., New York (1999).

Several screen schemes can be employed to screen for novel modulators of TMPRSS6. In some embodiments, test agents are first screened for binding to TMPRSS6. Agents thus identified are further tested for ability to modulate protease activity of the enzyme. In some other embodiments, test agents are directly screened for ability to alter proteolysis activity of a substrate by TMPRSS6. As noted above, the substrate can be either a pathogenic protein (bacterial or viral protein) that can be digested by TMPRSS6 or a synthetic substrate. Some methods are directed to identifying agents that inhibit activation of pathogenic proteins, and thereby treating infection of the pathogens that require proteolytic activation of their proteins (e.g., toxins) by TMPRSS6. In these methods, test agents are first screened for ability to bind to TMPRSS6 and/or ability to inhibit protease activity of the enzyme. Agents that have been identified to inhibit TMPRSS6 protease activity are then further examined for ability to inhibit infection of the pathogens.

A. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Examples of peptide libraries have been described in, e.g., Lam et al., Nature 354:82-84, 1991; Houghten et al., Nature 354:84-86, 1991), and Songyang et al., Cell 72:767-778, 1993. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be natural occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of TMPRSS6. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of TMPRSS6 or its fragments. Such structural studies allow the identification of test agents that are more likely to bind to TMPRSS6. The three-dimensional structure of TMPRSS6 or its fragments (e.g., its catalytic domain) can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of a target protein (e.g., TMPRSS6) provides another means for designing test agents for screening modulators of the target protein. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to TMPRSS6. Such antibodies can be monoclonal or polyclonal. Such antibodies can be generated using methods well known in the art. For example, the production of non-human monoclonal antibodies, e.g., murine or rat, can be accomplished by, for example, immunizing the animal with TMPRSS6 or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to TMPRSS6.

Human antibodies against TMPRSS6 can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using TMPRSS6 or its fragment.

B. Screening for Agents that Bind to TMPRSS6

In some methods, test agents are first screened for ability to bind to TMPRSS6. Typically, purified TMPRSS6, an enzymatic fragment, or an appropriate variant or analog is used in high-throughput screens to assay test agents for the ability to bind to the protease. Binding of test agents to TMPRSS6 can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. Agents that bind to TMPRSS6 can be identified by detecting a direct binding to TMPRSS6, e.g., co-immunoprecipitation with TMPRSS6 by an antibody directed to TMPRSS6. They can also be identified by detecting a signal that indicates that the agent binds to TMPRSS6, e.g., fluorescence quenching or FRET.

Competition assays provide a suitable format for identifying test agents that specifically bind to TMPRSS6. In such formats, test agents are screened in competition with a compound already known to bind to TMPRSS6. The known binding compound can be a synthetic compound. It can also be an antibody that specifically recognizes TMPRSS6, e.g., a monoclonal antibody directed against TMPRSS6. If the test agent inhibits binding of the compound known to bind TMPRSS6, then the test agent is also likely to bind TMPRSS6.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize TMPRSS6 or its fragment onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing TMPRSS6, the test agents are bound to the solid matrix and TMPRSS6 molecule is then added.

Soluble assays include some of the combinatory libraries screening methods described above. Under the soluble assay formats, neither the test agents nor TMPRSS6 are bound to a solid support. Binding of TMPRSS6 or fragment thereof to a test agent can be determined by, e.g., changes in fluorescence of either TMPRSS6 or the test agents, or both. Fluorescence may be intrinsic or conferred by labeling either component with a fluorophor.

In some binding assays, either TMPRSS6, the test agent, or a third molecule (e.g., an antibody against TMPRSS6) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group.

Binding of a test agent to TMPRSS6 provides an indication that the agent could be a modulator of the enzyme. A test agent that binds to TMPRSS6 can be further examined to determine its activity on the protease activity of the enzyme. The existence, nature, and extent of such activity can be tested by an activity assay as detailed below. Such an activity assay can confirm that the test agent binding to TMPRSS6 indeed has a modulatory activity on TMPRSS6. More often, as detailed below, such activity assays can be used independently to identify test agents that modulate activities of TMPRSS6 (i.e., without first assaying their ability to bind to TMPRSS6).

C. Screening for Agents that Modulate TMPRSS6 Protease Activity

In some methods, test agents are directly screened for ability to modulate proteolysis of a substrate by TMPRSS6. In some embodiments, the substrate employed is a pathogenic protein, e.g., a bacterial toxin. In some embodiments, the substrate is a synthetic peptide. Various assays can be employed to monitor effects of test agents on proteolysis of a substrate by TMPRSS6. Preferably, test agents are screened with a high-through screening format. Test agents that modulate protease activity of TMPRSS6 can be identified with both cell-based or cell-free assay systems. Cell-based systems can be native, i.e., cells that normally express the protease, e.g., endothelial cells. It is known that human TMPRSS6 is primarily expressed in lung, placenta, pancreas and prostate, but not in brain, heart, liver, intestine, kidney, thymus, muscle, or ovary (Kim et al., Biochimica et Biophysica Acta 1518: 204-209, 2001). Alternatively, cell-based assays involve recombinant host cells expressing the protease protein.

More often, cell-free systems are employed to screen for agents that alter (e.g., inhibit) the enzymatic activity of TMPRSS6. Typically, the assay system contains TMPRSS6 or an enzymatic fragment as described above, a labeled or un-labeled substrate (e.g., anthrax protective antigen or diphtheria toxin), as well as other reagents necessary for the enzymatic reaction (as exemplified in the Examples below). The enzyme is contacted with test agents prior to or concurrently with incubation with the substrate. Effect of the test agents on the protease activity is monitored by comparing digestion of the substrate in the reaction to that of a control reaction in which no test agent is present.

Methods for monitoring serine protease activity are well known in the art, e.g., as described in Sambrook et al. and Ausubel et al., supra. For example, proteolysis of Bacillus anthracis protective antigen can be assayed as described in, e.g., Beauregard et al., Cell. Microbiol. 2: 251-58, 2000; and Kim et al., Protein Expr Purif. 30(2):293-300, 2003. Specific methods are also disclosed in the art and in the present invention. In some methods, non-labeled substrates can be used. As exemplified in the Examples below, proteolysis of a bacterial toxin by TMPRSS6 can be monitored by electrophoresis followed by visualization of the reaction products. In some methods, proteolysis of an un-labeled substrate is monitored by zymography following SDS polyacrylamide gel electrophoresis (Wadstroem and Smyth, Sci. Tools 20: 17-21, 1973).

In other methods, a labeled substrate is used. Labeled substrates suitable for the screening include, e.g., substrates that are radio-labeled (Coolican et al., J Biol. Chem. 261: 4170-6, 1986; fluorometric (Lonergan et al., J Food Sci. 60:72-3, 78, 1995; and Twining, Anal. Biochem. 143: 30-4, 1984); or calorimetric (Buroker-Kilgore et al., Anal. Biochem. 208: 387-92, 1993). Effect of a test agent on digestion of the labeled substrate (e.g., a bacterial toxin) by the enzyme can be monitored by a number of means. In some preferred embodiments, the substrate is fluorescently labeled, and fluorescence signal due to the proteolysis is typically detected continuously, at multiple time points in the course of the enzymatic reaction, or at a single time point at or near the end of the reaction. By continually monitoring the fluorescence for each test agent, kinetic data can also optionally be obtained. For example, proteolysis of a fluorescently labeled substrate can be assayed as described in the Examples below. Briefly, a peptide substrate is labeled with the fluorophore 7-amino-4-carbamoylmethylcoumarin (acc). Protease activity of the purified TMPRSS6 polypeptide on the substrate was monitored by quantifying accumulation of the fluorescent signal due to the cleavage of the substrate.

In some other methods, fluorescent resonance energy transfer (FRET)-based methods (Ng et al., Anal. Biochem. 183: 50-6, 1989) can be employed in screening for agents that modulate TMPRSS6 protease activity. In some embodiments, FRET is used to detect cleavage of a labeled substrate. FRET is a distance dependent excited state interaction in which emission of one fluorophore is coupled to the excitation of another fluorophore which is in proximity, e.g., close enough for an observable change in emissions to occur. Typically, a FRET pair (a donor and an acceptor) are attached to the substrate on the two sides of the cleavage site. Once the substrate is cleaved, the donor and acceptor are no longer held in close proximity and the acceptor no longer quenches the donor signal. As a result, the donor then emits a signal that is observed by a detector. The detection can be monitored continuously or at multiple time points.

Many other assays for assaying protease activity of furin on bacterial toxins can also be employed to examine proteolysis of a substrate by TMPRSS6. Such assays have been described, e.g., in Molloy et al., J. Biol. Chem. 267: 16396-402, 1992; Klimpel et al., Proc Natl Acad Sci USA. 89: 10277-81, 1992; Chiron et al., J. Biol. Chem. 269: 18167-76, 1994; Tsuneoka et al., J Biol Chem. 268: 26461-5, 1993; and Gordon et al., Infect Immun. 4130-4, 1997.

In addition to assays for analyzing furin activity, there are also many known assays in the art for determining activities of other serine proteases. For example, Berdichevsky et al. (J Virol Methods. 107: 245-55, 2003) disclosed a high throughput screening assay for HCV NS3 serine protease inhibitors. This assay utilized purified NS3 serine protease and a substrate linked to a green fluorescent protein. Cleavage of the substrate results in emission of fluorescent light that is easily detected and quantitated by fluorometry. This screening assay was successfully employed to identify NS3 serine protease inhibitors from plant extracts. Another assay for screening for inhibitors of a serine protease was disclosed in Cho et al., J Virol Methods. 72: 109-15, 1998. This in vivo assay employs a substrate fused to secreted alkaline phosphatase. Activity of SEAP can be monitored quantitatively and continuously by the chemiluminescent method. Substituting with TMPRSS6 and similarly labeling one of its substrates (e.g., anthrax protective antigen), these assays can be readily used to screen for modulators (e.g., inhibitors) of TMPRSS6.

D. Examining TMPRSS6-Inhibiting Agents for Ability to Inhibit Infection of Pathogens

Once test agents have been identified to inhibit protease activity of TMPRSS6 on a synthetic peptide substrate or a pathogenic toxin, the agents can be subject to further analysis for ability to inhibit infection of pathogens in a subject. Such further analysis can be performed in a test animal (e.g., a mouse or rat) infected with a pathogen of interest (e.g., Bacillus anthracis). The analysis can be performed with an established animal model system for infection of a given pathogen. The animal is administered with an agent that inhibits TMPRSS6 protease activity. Control animals are administered with a compound that is known to have no antimicrobial activity. In some methods, the pathogen is allowed to grow for a set period of time, and the animal may then be sacrificed. Infected tissue is removed and the number of pathogen present in the infected tissue is determined using standard microbiological techniques.

Methods of using animal models for evaluating therapeutic activity of compounds on pathogenic infections are well known and routinely practiced in the art. For example, general principles and guidance for developing animal models for studying human infection are taught in Handbook of Animal Models of Infection: Experimental Models in Antimicrobial Chemotherapy, Sande (Ed.), Academic Press, 1st edition (Jun. 15, 1999); Animal Testing in Infectiology (Contributions to Microbiology, Vol. 9), Schmidt et al. (Eds.), S. Karger Publishing (December 2001); and Handbook of Laboratory Animal Science, Second Edition: Essential Principles and Practices, Volume I, Jann Hau et al. (Eds.), CRC Press, 2nd edition (Oct. 29, 2002).

In addition to the general knowledge and principles of employing animal models in studying activities of therapeutic compounds against human pathogens, the art has also disclosed specific methods and animal models for evaluating activities of potential therapeutics in treat infections of the pathogens described herein. For example, effects of TMPRSS6-inhibiting agents on infection of Bacillus anthracis can be examined with a mouse model. Mouse models for studying treatment of Bacillus anthracis have been disclosed in the art. For example, Thomas et al. (Antimicrob Agents Chemother. 46: 3463-71, 2002) disclosed antimicrobial therapy for Bacillus anthracis-induced polymicrobial infection in ⁶⁰Co-irradiated mice. A polymicrobial infection was developed in B6D2F1/J mice after nonlethal (7-Gy) ⁶⁰Co irradiation and intratracheal challenge with B. anthracis spores after irradiation. Effects of various antimicrobial agents on treating the mice were examined by monitoring survival of the mice and the response of the polymicrobial infection during the course of the therapy. Similarly, Welkos et al. (Microbiol. 147: 1677-85, 2001) disclosed use of antibodies to Bacillus anthracis and anthrax toxin components in inhibiting the early stages of infection by anthrax spores. These methods can all be easily modified and employed in the present invention to study effects of TMPRSS6-inhibiting agents on clearing Bacillus anthracis infection.

Other than Bacillus anthracis, effects of TMPRSS6-inhibiting agents on infections of other pathogens can also be examined. For example, activity of the agents on infection of Corynebacterium diphtheriae can be examined using methods described in the art, e.g., Wilson, J Antimicrob Chemother. 35: 717-20, 1995 which reviewed various schemes for treating infections caused by toxigenic and non-toxigenic strains of Corynebacterium diphtheriae. Additional art disclosing methods and therapeutics for treating diphtheria include, e.g., Smith et al., Br J Exp Pathol. 51: 73-80, 1970; Rub et al., Bangladesh Med Res Counc Bull. 15: 38-41, 1989; and Agarwal et al., J Indian Med Assoc. 79: 1-4, 1982. Animal models for evaluating therapeutic activity of compounds on Pseudomonas aeruginosa infection have been disclosed in the art, e.g., in Yasuda et al., Infect Immun. 58: 2502-9, 1990; Shapiro et al., J Antimicrob Chemother. 41: 403-5, 1998; Ng et al., Retina. 17: 464-5, 1997; Oishi et al., Antimicrob Agents Chemother. 37: 164-70, 1993; and Tsuchimori et al., J Antimicrob Chemother. 39: 423-5, 1997. For Shigella dysenteriae infection, the art has also disclosed several animal models for studying activities of therapeutic or prophylactic agents. See, e.g., Gendrel et al., Clin Infect Dis. 24: 83, 1997; Venkatesan et al., Infect Immun. 70: 2950-8, 2002; and Eiklid et al., J Immunol. 130: 380-4, 1983.

V. Therapeutic Applications

The present invention provides compositions and methods for treating infections of pathogens that require proteolytic activation of their proteins by a host furin-like protease (e.g., TMPRSS6). For example, there are a number of bacterial infections that are amenable to treatment with the TMPRSS6 inhibitors of the present invention. As noted above, these include infections of Bacillus anthracis, Pseudomonas aeruginosa, Corynebacterium diphtheriae, and Shigella dysenteriae. The compositions and methods can also be used in the treatment of certain viral infections, e.g., infections of HTLV-II, Semliki Forest virus, and Molluscum contagiosum virus. Inhibition of TMPRSS6 protease activity can also be useful for preventing or modulating the development of such infections in a subject (e.g., human or non-human mammals) of being, or known to be, prone to infections of these pathogens.

The TMPRSS6-inhibiting agents can be administered directly to a subject that is infected by a pathogen (e.g., Bacillus anthracis). The modulators can be administered alone or as the active ingredient of a pharmaceutical composition. Administration can be by any of the routes which are well known to those of skill in the art and which are normally used for introducing a modulating compound into ultimate contact with the tissue to be treated.

The TMPRSS6-inhibiting modulators of the present invention can be administered to a subject at therapeutically effective doses to prevent, treat, or control diseases or conditions associated with infections of the various pathogens. The compounds are administered to a subject in an amount sufficient to elicit an effective protective or therapeutic response in the subject. An effective protective or therapeutic response is a response that at least partially arrests or slows the symptoms or complications of the disease. An amount adequate to accomplish this is defined as “therapeutically effective dose.” The optimal dose level for any subject will depend on a variety of factors including the efficacy of the specific modulator employed, the age, body weight, physical activity, and diet of the subject, and on a possible combination with other drugs. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicity, and possibly the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in treating or preventing human infection by any of the pathogens described herein. The modulators of the invention and another agent may be co-administered, either in concomitant therapy or in a fixed combination, or they may be administered at separate times. There are many known antimicrobial agents that can be employed in the present invention. For example, as detailed in Example 3 below, camostat mesylate (Sequoia Research Products Ltd., Oxford, UK) was able to inhibit TMPRSS6 protease activity. To treat or prevent infection of the bacterial pathogens, the therapeutic or prophylactic scheme can employ this compound alone or in combination other novel TMPRSS6-inhibiting agents of the present invention. Various other known inhibitors of serine protease or furin may also be used to practice methods of the present invention. Example of such inhibitors have been described in, e.g., Villemure et al., Biochemistry 42: 9659-9668, 2003; Sarac et al., Infect Immun. 70: 7136-9, 2002; Dahlen et al., J Biol Chem. 273: 1851-4, 1998; U.S. Pat. No. 5,900,400; and U.S. Pat. No. 5,807,829.

The pharmaceutical compositions of the invention may comprise a pharmaceutically acceptable carrier. Pharmaceutically acceptable carriers are determined in part by the particular composition being administered, as well as by the particular method used to administer the composition. There are a wide variety of suitable formulations of pharmaceutical compositions of the present invention (see, e.g., Remington: The Science and Practice of Pharmacy, Mack Publishing Co., 20^{th} ed., 2000).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

### EXAMPLES

The following examples are presented to further illustrate certain aspects of the invention and are not to be construed so as to limit the scope of the invention.

### Example 1

### Expression and Purification of TMPRSS6 Protease

The putative catalytic domain of TMPRSS6 minus the pro region was cloned into pFastBac1 (Invitrogen) from a human cDNA library. The sequence was found to be 100% identical to the corresponding catalytic region of the reported sequence (accession number AY190317). The vector also contains a C-terminal 6His tag to facilitate purification and an N-terminal honeybee melittin secretion signal. Recombinant bacmid was produced using Bac-to-Bac transposition technology (Invitrogen). Sf9 cells (Invitrogen) were then transfected with recombinant bacmid to produce rAcNPV.

Expression of the polypeptide was done in 1L vented shake flasks (Corning) in serum free insect medium (Expression Systems). Cells were cultured to a density of 10⁶/ml and infected at an MOI of 0.1. After 48 hours incubation, the culture medium was harvested for processing. The pH of the medium was raised to 8.0 by the addition of 0.5M Tris-Cl pH 9.0 to precipitate detergent which was later removed by centrifugation. The medium was diafiltrated against 10 volumes of 50 mM NaPO₄ 50 mM NaCl pH 7.4. NaCl was added to 300 mM and the retentate was then filtered and passed over a cobalt IMAC column (BDClontech) at 1 ml/min. The column was washed with several column volumes of 50 mM NaPO₄ 300 mM NaCl pH 7.4. Bound material was eluted with a gradient of 0 to 150 mM imidazole in 50 mM NaPO₄ 300 mM NaCl pH 7.4. The active fractions were pooled and dialyzed against PBS pH 7.4. The dialyzed samples were then concentrated by ultrafiltration and stored at 4 C. For further purification, some of the active material was passed over a superdex 200 size exclusion column (Amersham) equilibrated with PBS pH 7.4. The active fractions were pooled and analyzed by SDS-PAGE.

### Example 2

### Protease Activity and Substrate Specificity of TMPRSS6

This Example describes characterization of TMPRSS6, including its substrate specificity and kinetics of its protease activity. The substrate specificity of a protease is an important characteristic that often governs its biological activity. Libraries of substrates and methods for profiling substrate specificity of an enzyme (e.g., determining its recognition sequences) have been described in detail in the art, e.g., WO 03/029823.

Protease activity of the purified TMPRSS6 was assessed by monitoring the accumulation of the fluorophore 7-amino-4-carbamoylmethylcoumarin (acc) produced by the cleavage of the tetrapeptide substrate Ac-RKFK-acc in assay buffer (50 mM Tris-Cl pH 7.4, 200 mM NaCl, 2 mM CaCl₂, 0.01% Tween 20) at 37 C. Data was collected with a Gemini EM plate reader (Molecular Devices) at λₑₓ=380 nm and λₑₘ=450 nm.

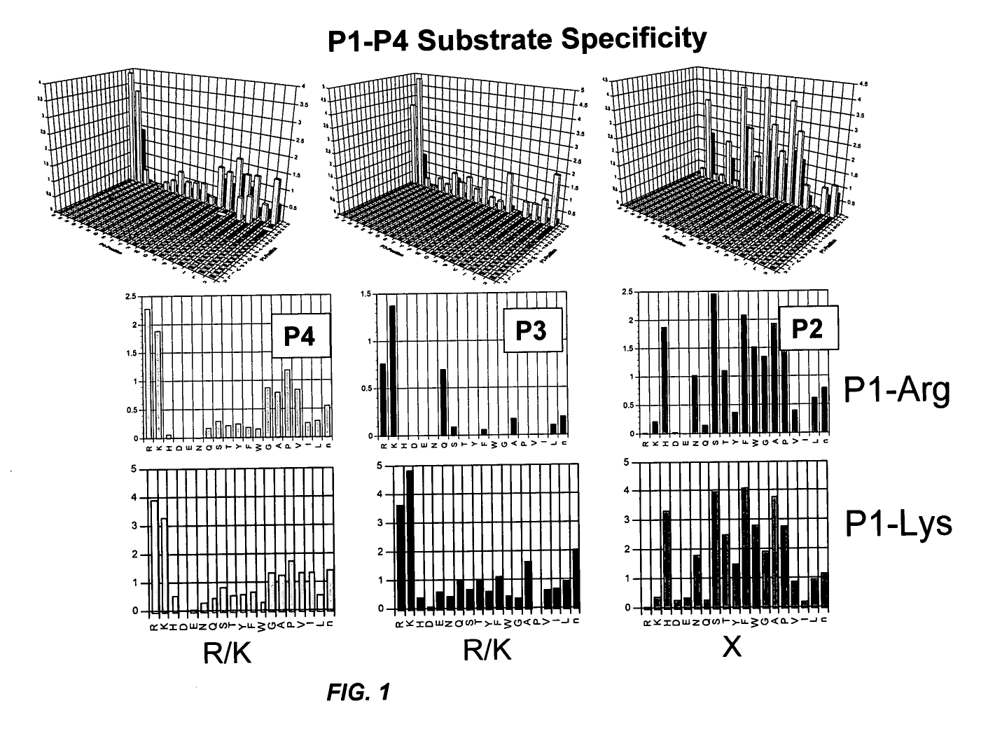

Optimal substrate was then determined. The substrate specificity was identified using a positional scanning synthetic combinatorial tetra-peptide library. For the P1-P4 positions, fluorogenic substrate libraries were synthesized as described in Wang et al, J. Biol. Chem., 278: 15800-15808, 2003. The enzyme was diluted in assay buffer and added to the library plates. In the two-position fixed tetrapeptide substrate library, the final substrate concentration was approximately 0.25 μM/substrate/well with a total of 361 substrates per well. Each variable position had one of 19 amino acids with cysteine excluded and methionine replaced by the isosteric amino acid norleucine. Accumulation of fluorophore was monitored at 37 C at a λₑₓ of 380 nm and a λₑₘ of 450 nm with a Gemini XS plate reader (Molecular Devices) (FIG. 1).

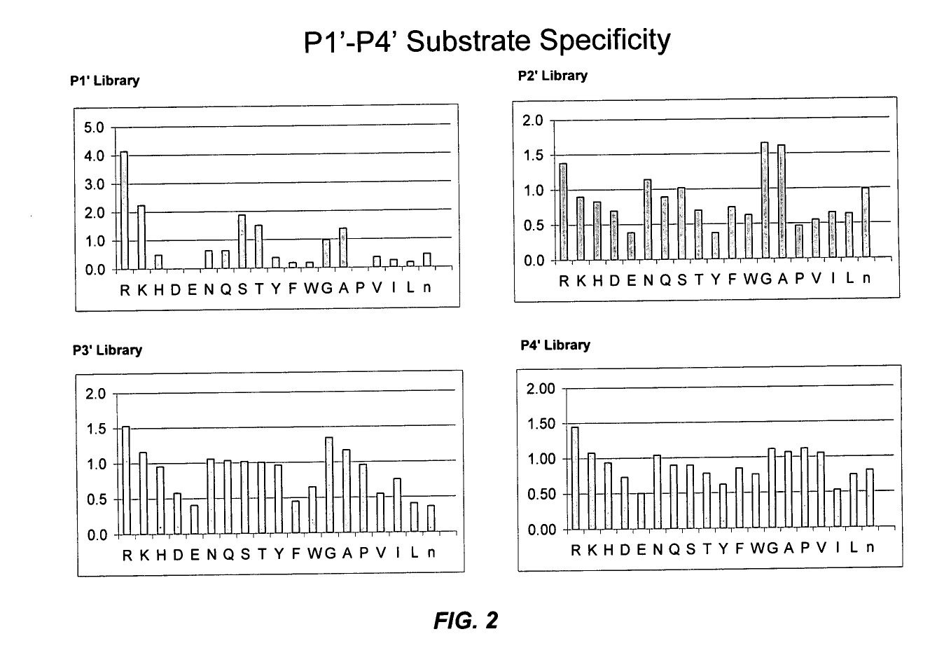

An optimal substrate sequence, RKFK (from the N-terminus to the C-terminus), was chosen based on the results of the P1-P4 libraries and used in the synthesis of a focused donor-quencher library for the elucidation of the P1′-P4′ substrate specificity of TMPRSS6. An 80 well library was first synthesized using split and mix technology where each well contained a tetrapeptide sequence with one fixed and three variable amino acids followed by a lysine modified with a di-nitrophenyl quencher group and an arginine for enhanced solubility. Each fixed position was composed of one of 20 amino acids (excluding cysteine and including both methionine and its isostereo norleucine). Following the P1′ position of the hexapeptide library, the optimal non-prime sequence, RKFK, was synthesized and acylated with a fluorogenic coumarin donor group. After cleaving the substrates from a solid support, the library was lypophilized and dissolved in DMSO. For kinetic assays, 1 μl of the reconstituted library was added to 99 μl of assay buffer (see “Enzyme assays”) containing TMPRSS6. The final concentration of each substrate in the assay was approximately 4 nM. The increase in fluorescence intensity was measured over time at a λₑₓ of 320 nm and a λₑₘ of 380 nm with a Gemini XS plate reader (Molecular Devices) (FIG. 2).

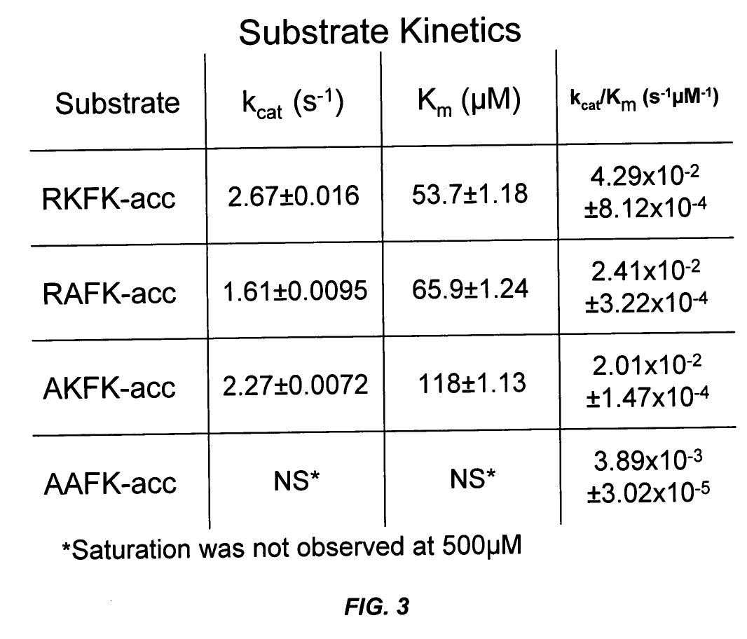

Kinetics of TMPRSS6 on a substrate is shown in FIG. 3. Based on the substrate specificity profile, the optimal substrate Ac-RKFK-acc was synthesized. Ac-AAFk-acc, Ac-AKFK-acc, and Ac-RAFK-acc were also synthesized to determine the importance of the P3 and P4 positions for specificity. 100× solutions in DMSO were made for each substrate covering a range of concentrations (50.0-0.1 mM). Enzyme was diluted in assay buffer to a final concentration of 1 to 10 nM in 99 μl of substrate was added and the reactions were monitored as described under “Enzyme assays.” The data were fit by non-linear regression to the Michelis-Menten equation and k_{cat}, Km and k_{cat}/Km were calculated.

### Example 3

### Inhibition of TMPRSS6 Protease Activity by Camostat Mesylate

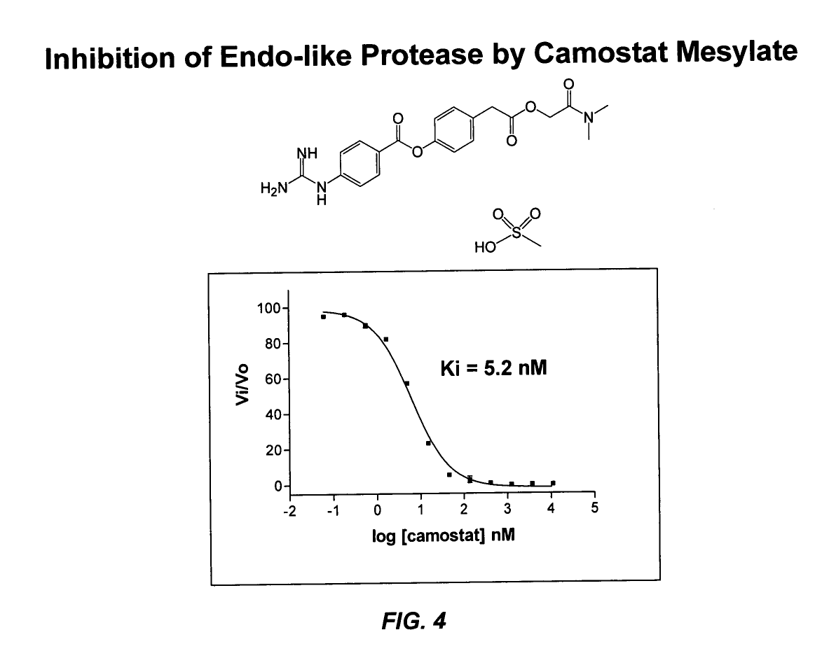

FIG. 4 shows the results of a study on inhibition of TMPRSS6 protease activity by camostat mesylate. A 1 mM DMSO solution of camostat mesylate was diluted serially 1:3 in DMSO to a final concentration of 5.6 nM. The TMPRSS6 enzyme was diluted in assay buffer to 2 nM. 1 μl of each camostat dilution was added to 98 μl of the enzyme mixture in a 96 well assay plate (Dynex). The plate was incubated for 15 min at room temperature to allow equilibrium to take place between the enzyme and inhibitor. Afterward, 1 μl of a 1 mM solution of a substrate, Ac-RKFK-acc, was added to each well. Kinetic data was collected as described under above. The inhibition constant was calculated using Prism (GraphPad).

These data indicate that the protease activity of the TMPRSS6 enzyme is inhibited by camostat mesylate.

### Example 4

### Cleavage of Bacterial Toxins by TMPRSS6

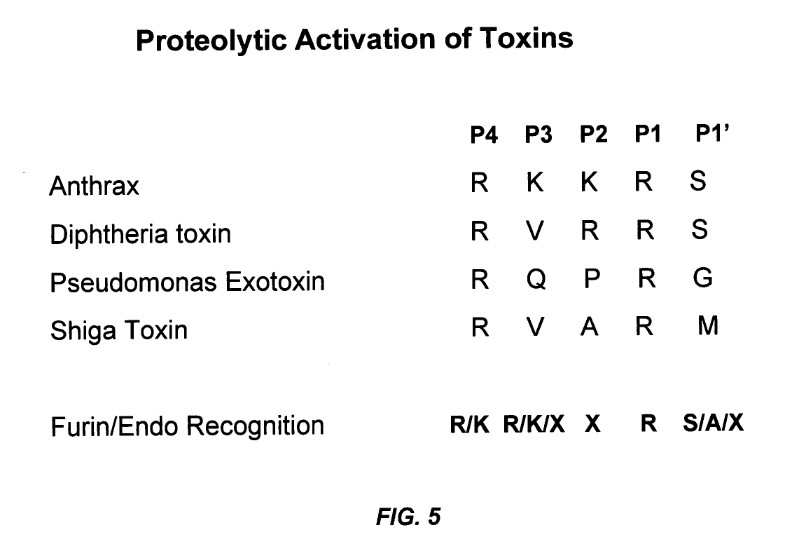

Many bacterial toxins require activation by proteolytic cleavage. The substrate specificity of TMPRSS6 resembles the known activation sites of several of these toxins recognized by furin (FIG. 5). This example describes enzymatic digestion of purified anthrax protective antigen and diphtheria toxin by TMPRSS6.

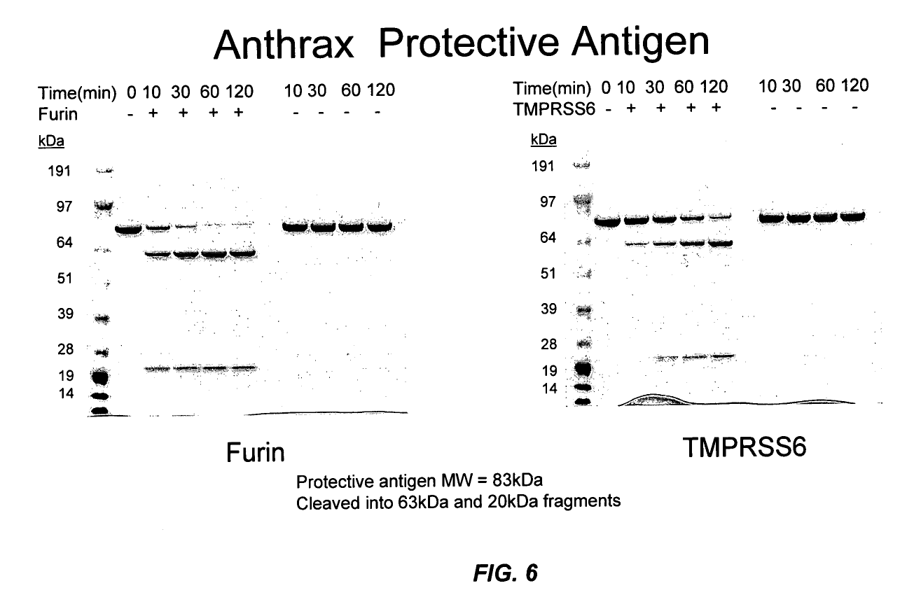

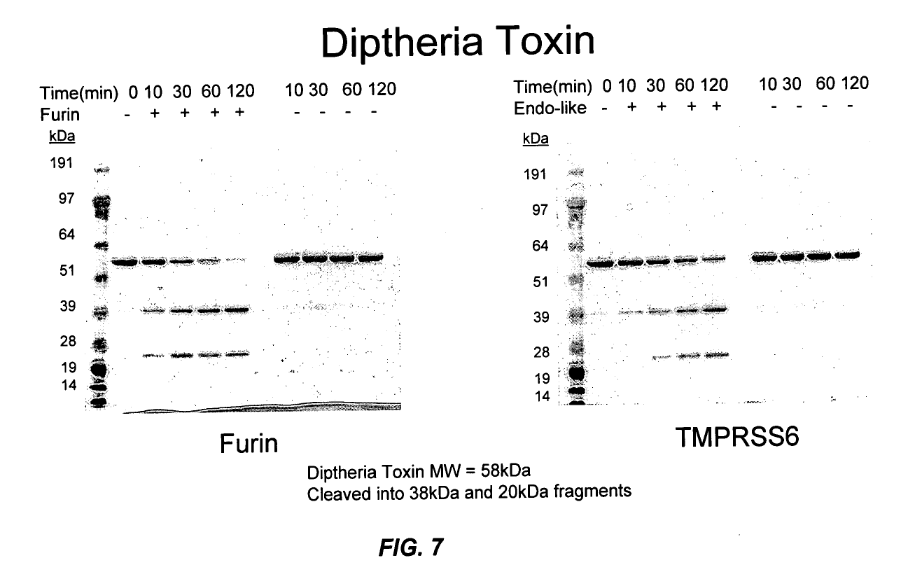

Intact diphtheria toxin of Corynebacterium diptheriae (DT) and intact protective antigen of Bacillus anthracis (PA) were obtained from List Biological Labs, Inc. Toxin digests were set up with 1 μM toxin and 50 nM TMPRSS6 protease or 5 nM furin (New England Biolabs) in assay buffer. Control reactions contained toxin without enzyme. Reactions were incubated at 37 C for 2 hours. At 0, 10, 30, 60 and 120 minutes a 20 μl sample was removed and mixed with 6.7 μl 4×LDS sample buffer (Invitrogen) containing 400 mM 2-mercaptoethanol and kept on ice. At the end of the incubation, the samples were heated at 70 C for 10 min and resolved by SDS-PAGE on a 4-12% Bis-Tris NuPAGE gel in MOPS buffer (Invitrogen). After electrophoresis at 200V for 45 min., the gels were fixed in 40% methanol/10% acetic acid and stained with coomassie G250 (Gel Code-Pierce) (FIGS. 6-7).

The results suggest that TMPRSS6 could play a role in activation of the various pathogenic toxins under physiological conditions.

### Example 5

### Cytotoxicity of a Toxin Cleaved by TMPRSS6

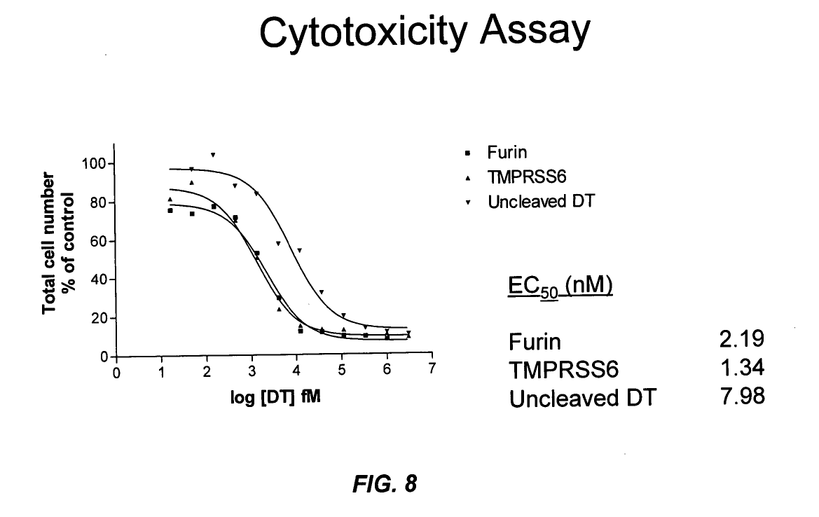

Cytotoxicity of cleaved diphtheria toxin (DT) was determined with a Vero cell assay (Sandivg and Olsens, J. Biol. Chem. 256: 9068-9076, 1981). Vero cells were obtained from ATCC and cultured in growth medium (MEM, 2 mM GlutaMax, 1 mM sodium pyruvate, 0.1 mM nonessential amino acids, 10% fetal bovine serum). All cell culture reagents were obtained from Invitrogen Corporation. 24 well cell culture plates (Corning) were seeded with 5×103 cells/well in growth medium. Nicked diphtheria toxin, produced by 4 hours digestion with 5 nM furin or 100 nM TMPRSS6 as previously described, was kept on ice until the time of the assay. After 48 hours incubation, the cells were washed once with binding buffer (MEM, 2 mM GlutaMax, 1 mM sodium pyruvate, 0.1 mM nonessential amino acids) and various concentrations of digested or undigested toxin, diluted in binding buffer were added to the wells in a volume of 0.5 ml. The cells were then incubated at 37° C. for 30 min. Afterward, the toxin was removed and growth medium was added to the wells. After 36 hours, the medium was removed and 250 μl of a 1× cell lysis solution was added to the wells. After 30 min. incubation at 37 C, the samples were assayed for total LDH (lactate dehydrogenase) activity to quantitate the total number of cells per well (Promega Cytotox 96 Assay Kit). The results were compared to control values and plotted in Prism. As shown in FIG. 8, in wells with low nM and high pM concentrations of furin- or TMPRSS6-cleaved toxin, there was extensive cell death. The corresponding wells containing uncleaved toxin contained relatively few dead cells.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

1. A method for identifying an agent that inhibits proteolytic activation of a toxin of a pathogen, the method comprising (a) assaying proteolysis of the toxin by transmembrane protease serine 6 (TMPRSS6), or an enzymatic fragment of TMPRSS6, in the presence of test agents; and (b) identifying a test agent that inhibits proteolysis of the toxin by TMPRSS6.

2. The method of claim 1, wherein the pathogen is selected from the group consisting of Bacillus anthracis, Pseudomonas aeruginosa, Corynebacterium diphtheriae, and Shigella dysenteriae.

3. The method of claim 1, wherein the toxin is anthrax protective antigen of Bacillus anthracis.

4. The method of claim 1, wherein the toxin is pseudomonas exotoxin of Pseudomonas aeruginosa.

5. The method of claim 1, wherein the toxin is diphtheria toxin of Corynebacterium diphtheriae.

6. The method of claim 1, wherein the toxin is shiga toxin of Shigella dysenteriae.

7. The method of claim 1, wherein the enzymatic fragment of TMPRSS6 comprises the catalytic domain of the enzyme.

8. The method of claim 1, wherein TMPRSS6 is encoded by a polynucleotide having accession number AY190317, AB048797 or AB048796.

9. The method of claim 1, wherein proteolysis of the toxin is examined by polyacrylamide gel electrophoresis.

10. A method for identifying an agent that inhibits infection of a pathogen, the method comprising (a) assaying protease activity of transmembrane protease serine 6 (TMPRSS6), or an enzymatic fragment of TMPRSS6, in the presence of test agents to identifying one or more modulating agents that inhibit the protease activity of TMPRSS6, and (b) examining the modulating agents for ability to inhibit or clear infection of the pathogen.

11. The method of claim 10, wherein the pathogen is selected from the group consisting of Bacillus anthracis, Pseudomonas aeruginosa, Corynebacterium diphtheriae, and Shigella dysenteriae.

12. The method of claim 10, wherein the enzymatic fragment of TMPRSS6 comprises the catalytic domain of the enzyme.

13. The method of claim 10, wherein TMPRSS6 is encoded by a polynucleotide having accession number AY190317, AB048797 or AB048796.

14. The method of claim 10, wherein protease activity of TMPRSS6 is assayed with a toxin of the pathogen.

15. The method of claim 10, wherein protease activity of TMPRSS6 is assayed with a synthetic peptide substrate.

16. The method of claim 15, wherein sequence at P4-P1 positions of the peptide substrate is selected from the group consisting of RKFK, RAFK, AKFK, and AAFK.

17. The method of claim 15, wherein the peptide substrate is labeled with fluorogenic compound.

18. The method of claim 17, wherein the fluorogenic compound is 7-amino-4-carbamoylmethylcoumarin or 7-amino-3-carbamoylmethyl-4-methylcoumarin.

19. The method of claim 10, wherein the modulating agents are examined with an animal infected with the pathogen.

20. A method for treating infection of a pathogen in a subject, the methods comprising administering to the subject a pharmaceutical composition comprising an effective amount of an agent that inhibits the protease activity of transmembrane protease serine 6 (TMPRSS6).

21. The method of claim 20, wherein TMPRSS6 is encoded by a polynucleotide having accession number AY190317, AB048797 or AB048796.

22. The method of claim 20, wherein the pathogen is selected from the group consisting of Bacillus anthracis, Pseudomonas aeruginosa, Corynebacterium diphtheriae, and Shigella dysenteriae.

23. The method of claim 20, wherein the agent is camostat mesylate.

24. The method of claim 20, wherein the agent is identified in accordance with claim 1.

25. The method of claim 24, wherein the pathogen is Bacillus anthracis, and the toxin is anthrax protective antigen.

26. The method of claim 24, wherein the pathogen is Corynebacterium diphtheriae, and the toxin is diphtheria toxin.

27. The method of claim 20, wherein the agent is identified in accordance with claim 10.

28. The method of claim 27, wherein the pathogen is Bacillus anthracis.

29. The method of claim 27, wherein the pathogen is Corynebacterium diphtheriae.
